(19) United States
(12) Patent Application Publication
(10) Pub. No.: US 2011/0080991 A1
(43) Pub. Date: Apr. 7, 2011

(54) X-RAY DEVICE FOR CONTROLLING A DC-AC CONVERTER

(75) Inventors: Thomas Scheel, Stolberg (DE); Christian Hattrup, Wurselen (DE); Max Urban, Hamburg (DE); Peter Kratsch, Henstedt-ulzburg (DE)

(73) Assignee: Koninklijke Philips Electronics N.V., Eindhoven (NL)

(21) Appl. No.: 12/994,468

(22) PCT Filed: May 27, 2009
(86) PCT No.: PCT/IB2009/052223

§ 371 Date: Nov. 24, 2010

(30) Foreign Application Priority Data
Jun. 2, 2008 (EP) 08104195.6

(51) Int. Cl. A61B 6/03; H05G 1/12

(52) U.S. Cl. 378/4; 378/104

(57) ABSTRACT

The invention provides an X-ray device for controlling a DC-AC converter, wherein the DC-AC converter is adapted for supplying a resonant circuit and a transformer (105) of a computer tomography gantry (91) with electrical energy, wherein the gantry comprises a rotary part (93) and a stationary part (92), wherein the transformer (105) is adapted for providing a current, feeding a high voltage rectifier circuit (106), providing an output voltage (107), the X-ray device comprises a detector for detecting the output voltage, a predictor (501) for calculating a first output with the use of processing the output voltage (107), wherein the first output represents the change of the output voltage (107) for the possible states of the DC-AC converter (102), a control loop (503) for calculating the required change of the output voltage (107) with the use of processing the output voltage (107) and the target specification, a decision block (502) for calculating a control value with the use of processing the first output and the required change of the output voltage (107), wherein the detector, the predictor (501), the control loop (503) and at least a part of the decision block (502) are adapted to be mounted on the rotary part of the gantry (93), such as the information content to be transmitted from the rotary part of the gantry (93) to the stationary part of the gantry (92) is less than the information content of the output voltage. A further aspect of the invention is a computer tomography gantry (91) comprising an X-ray device according to the invention.

### FIELD OF THE INVENTION

The present invention relates to a X-ray device for controlling a DC-AC converter. Further, the present invention relates to a computer tomography gantry comprising an X-ray device for controlling a DC-AC converter.

### BACKGROUND OF THE INVENTION

Novel computer tomography systems with very high output power have an architecture, wherein the components of the high voltage generator are placed on the rotary part of the gantry as well as on the stationary part of the gantry. Especially the DC-AC converter is arranged on the stationary part of the gantry, while the rectifier and the tube are mounted on the rotary part of the gantry. The energy can be transferred via a rotary transformer.

Due to this arrangement control signals have to be transferred from the rotary part of the gantry, where the tube is situated, to the stationary part of the gantry, where the DC-AC converter is placed. For the data transfer a contactless data link is preferred as it increases reliability and maintenance costs, compared to a solution using electrical connections.

### SUMMARY OF THE INVENTION

The speed of information transfer within the computer tomography gantry determines as one criterion the quality of the output voltage, and thus the image quality. The data transfer between the rotary part of the gantry and the stationary part of the gantry can be considered as bottleneck. Therefore, this part of the data transfer has to be optimized in order to accelerate the data processing.

It would be desirable to provide an improved device for optimizing the data transfer between the rotary part of the gantry and the stationary part of the gantry.

The invention provides an X-ray device for controlling a DC-AC converter, wherein the DC-AC converter is adapted for supplying a resonant circuit and a transformer of a computer tomography gantry with electrical energy, wherein the gantry comprises a rotary part and a stationary part, wherein the transformer is adapted for providing a current, feeding a high voltage rectifier circuit, providing an output voltage, the X-ray device comprises a detector for detecting the output voltage, a predictor for calculating a first output with the use of processing the output voltage, wherein the first output represents the change of the output voltage for the possible states of the DC-AC converter, a control loop for calculating the required change of the output voltage with the use of processing the output voltage and the target specification, a decision block for calculating a control value with the use of processing the first output and the required change of the output voltage, wherein the detector, the predictor, the control loop and at least a part of the decision block are adapted to be mounted on the rotary part of the gantry, such as the information content to be transmitted from the rotary part of the gantry to the stationary part of the gantry is less than the information content of the output voltage.

The invention provides a possibility to disburden the bottleneck of data transfer for the control signals of a computer tomography gantry. This bottleneck is the interface between the rotary part of the gantry and the stationary part of the gantry.

The invention provides also a computer tomography gantry comprising an X-ray device according to one of the claims 1 to 10.

Further embodiments are incorporated in the dependent claims.

According to the present invention an X-ray device is provided, wherein the control loop is a PI-control loop.

According to another exemplary embodiment an X-ray device is provided, wherein the decision block is adapted to calculate the control value to control the DC-AC converter.

According to the present invention an X-ray device is provided, further comprising a logic unit for controlling the DC-AC converter, wherein the decision block is adapted to control the logic unit by the control value.

According to another exemplary embodiment an X-ray device is provided, wherein the logic unit is programmable.

According to another exemplary embodiment an X-ray device is provided, wherein the logic unit is a FPGA or a CPLD.

According to an exemplary embodiment an X-ray device is provided, wherein the logic unit is adapted to be mounted on the stationary part of the gantry.

According to the present invention an X-ray device is provided, wherein the predictor is adapted for generating three predictions of the required change of the output voltage.

According to an exemplary embodiment an X-ray device is provided, wherein the predictor is adapted for generating five predictions of the required change of the output voltage.

According to an exemplary embodiment an X-ray device is provided, wherein the resonant circuit comprises a resonance capacitor, wherein the X-ray device comprises a second detector for detecting the capacitor voltage over the resonance capacitor, wherein the predictor is adapted for calculating a second output with the use of processing the capacitor voltage, which represents the change of the output voltage for the possible states of the DC-AC converter.

It may be seen as a gist of the present invention to provide an X-ray device, which minimizes the amount of control data that has to be transferred between the rotary part of the gantry and the stationary part of the gantry. This X-ray device renders the possibility for a high precision and accelerated data processing of the computer tomography gantry.

It should be noted that the above features may also be combined. The combination of the above features may also lead to synergetic effects, even if not explicitly described in detail.

These and other aspects of the present invention will become apparent from and elucidated with reference to the embodiments described hereinafter.

### BRIEF DESCRIPTION OF THE DRAWINGS

Exemplary embodiments of the present invention will be described in the following with reference to the following drawings.

FIG. 1 shows a functional diagram of a high voltage generator,

FIG. 2 shows a switch configuration of a DC-AC converter for plus state,

FIG. 3 shows a switch configuration of a DC-AC converter for 0 state,

FIG. 4 shows a switch configuration of a DC-AC converter for minus state,

FIG. 5 shows a structure of a controller for a three-level control,

FIG. 6 shows an application of energy levels for arbitrary operating points,

FIG. 7 shows an embodiment of a decision block,

FIG. 8 shows the method of operation of a decision block,

FIG. 9 shows a computer tomography gantry.

### DETAILED DESCRIPTION OF EMBODIMENTS

This invention is in particular intended for the use in a computer tomography system. Such a computer tomography system comprises a rotary part of the gantry, where the tube is mounted, and a stationary part of the gantry. Typically, the complete x-ray generator, comprising the DC-AC converter, the transformer and the rectifier, is placed on the rotary part of the gantry. The 3-phase mains are transferred via slip rings.

A control method is known, which allows zero current switching for all operating points while maintaining full controllability of the output voltage. Due to the zero current switching the power losses are very low. The control method is also very robust and has extraordinary good dynamic behaviour.

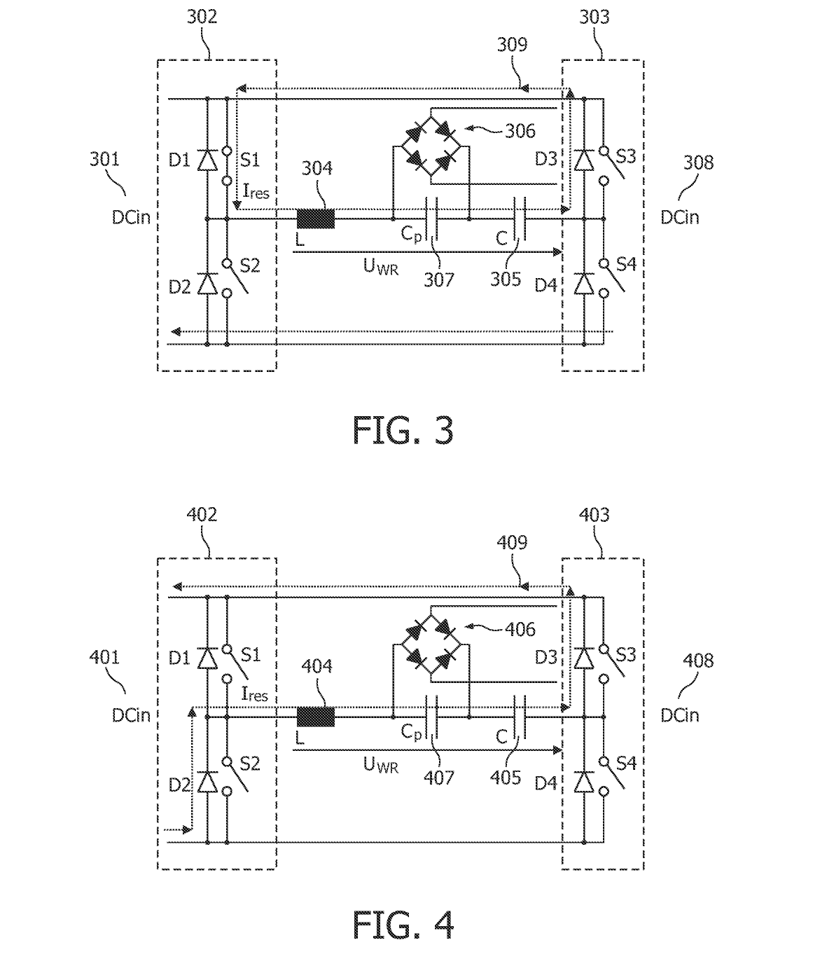

This control method is based on the transfer of discrete energy portions from the mains supply to the output. The discrete energy portions are generated by applying a voltage to the resonant circuit that is in-phase to the current (+ state, see FIG. 2), a zero voltage (0 state, see FIG. 3) or a voltage that is in anti-phase relation to the current (− state, see FIG. 4).

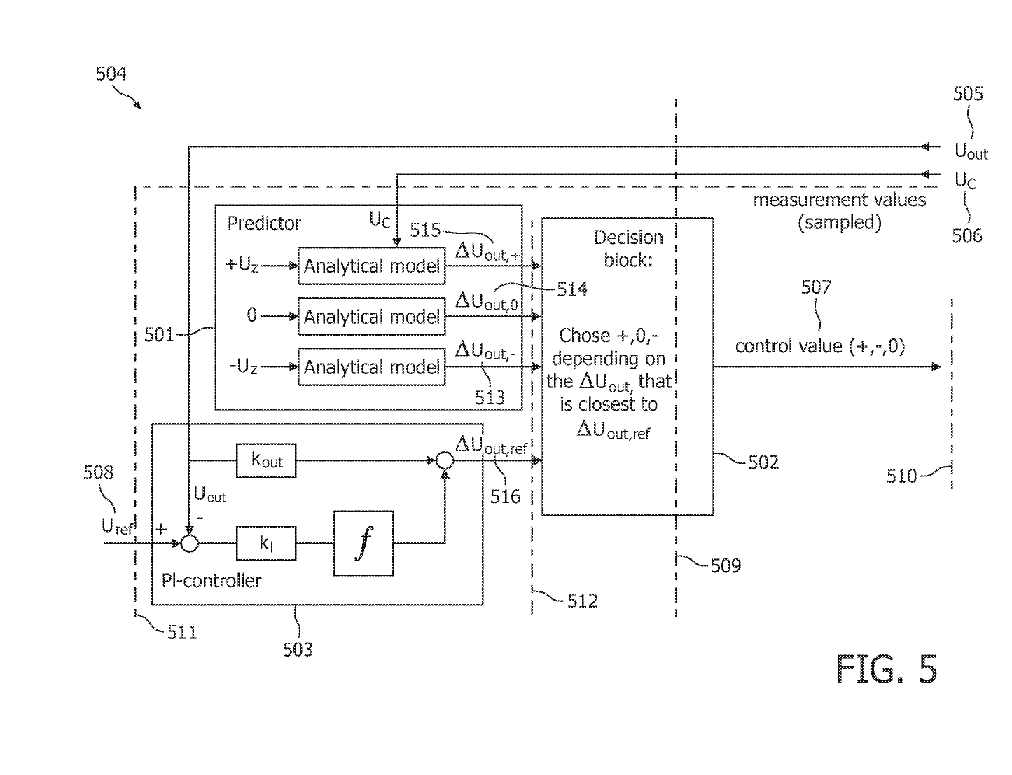

The decision which of the three operation modes (+,−,0) should be applied at a certain zero crossing of the resonant current is executed by a three-level controller 504 depicted in FIG. 5.

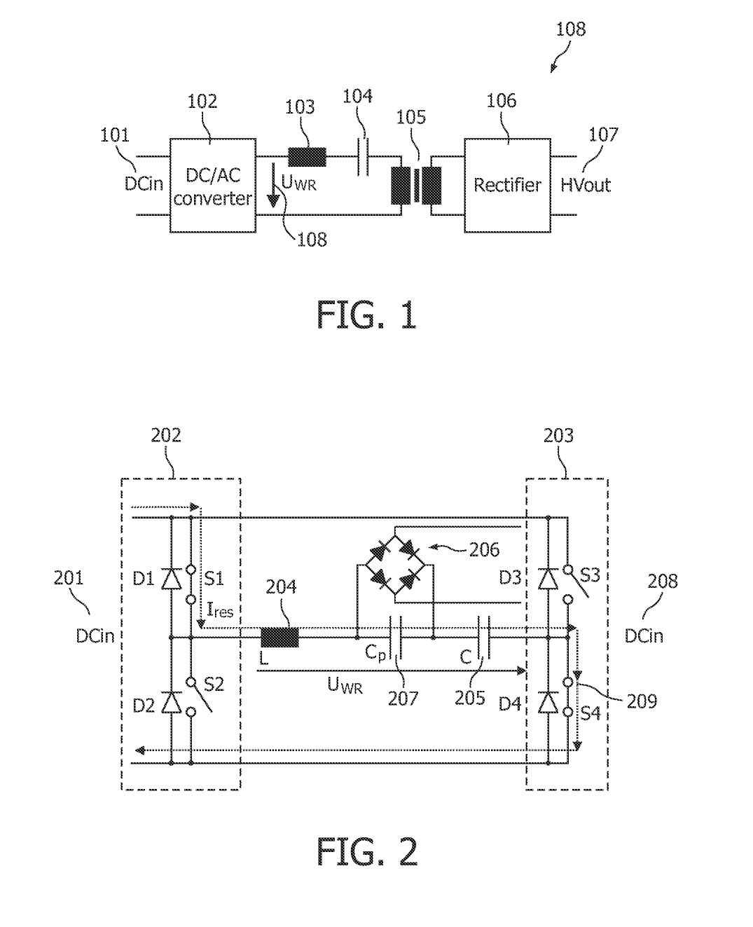

FIG. 1 shows a functional diagram of a high voltage generator 108. A DC input voltage 101 is converted in an AC voltage 108, which is fed to a resonant circuit 103, 104 and a primary side of a transformer 105. The output voltage of the transformer 105 is rectified by rectifier 106, which leads to a high voltage output 107. The resonant circuit 103, 104 comprises an inductance 103 as well as a capacitor 104. The leakage inductance of the transformer 105 can be a part of the resonant inductance 103. The leakage inductance of the transformer 105 can also replace the resonant inductance 103 totally.

FIG. 1 shows a functional diagram of a high voltage generator 108, which is realized as series resonant converters. A DC input voltage 101 is converted to an AC voltage 108 and fed into a series resonant circuit that compensates for the leakage inductivity of the transformer 105. The high voltage transformer 105, which is part of the series resonant circuit, transforms the low voltage (e.g. 400V) into a high voltage (e.g. 40 kV). Finally a rectifier 106 rectifies the output voltage of the transformer 105. The rectifier can comprise a cascade, which increases the voltage and generates a high DC voltage 107.

The DC-AC converter is typically realized as a full bridge converter, as depicted in the FIGS. 2, 3 and 4.

FIG. 2 shows a full bridge converter, which is realized as a one-phase bridge. The DC-AC converter comprises the blocks 202 and 203. These blocks 202, 203 are supplied by the DC input voltage 201, 208. The DC input voltage 201, 208 will be switched by the power switches S1, S2, S3 and S4. The switched voltage will be supplied to the resonant circuit 204 and 205 and to the transformer 207. In the FIG. 2 the transformer is represented by the parasitic capacitor 207. At the secondary side of the transformer 207 it is provided a rectifier 206, whose output voltage is the high voltage output 107. The diodes D1, D2, D3 and D4 are free-wheeling diodes. The situation in FIG. 2 is called the plus state. The rectifier 206 comprises four diodes, which rectify the output voltage of the transformer 207.

The current 209 of FIG. 2 shows the normal situation. In this situation the DC-AC converter supplies the resonant current with the inductance 204 and the resonant capacitor 205 and the transformer 207 with electrical energy. Voltage and current are in-phase (+ state).

FIGS. 3 and 4 show the same elements as in FIG. 2. The difference between these three figures (FIG. 2, FIG. 3 and FIG. 4) is the situation of the switching elements S1, S2, S3 and S4.

FIG. 3 shows the situation when three of the four switches S1, S2, S3 and S4 are in the off-state (not conducting). In this situation the free-wheeling diodes D1, D2, D3, D4 together with the switch in the on-state enables the flow of a current 309.

FIG. 3 shows the DC input voltage 301 and 308, which is fed to the DC-AC converter 302 and 303. The DC-AC converter 302 and 303 comprises four free-wheeling diodes D1, D2, D3 and D4. Further, the DC-AC converter 302, 303 comprises the four switches S1, S2, S3 and S4 for switching the input voltage 301, 308. These switched voltage is supplied to the resonant circuit with the inductance 304 and the capacitor 305 and the transformer 307. The transformer 307 is represented by the parasitic capacitor CP. The output voltage of the transformer 307 is fed to the rectifier 306, which is realized by four one-way conducting elements. These one-way conducting elements could be for example realized by four diodes or a rectifier cascade, wherein the rectifier rectifies the voltage and the cascade increases the voltage. FIG. 3 shows one situation, wherein three of the four switches are in an off-state. The situation of FIG. 3 is only one possibility for positive current. The other possible situation, which is not depicted in FIG. 3 is that the switch S4 is closed and the switches S1, S2 and S3 are in a off-state. For negative current switches S2 and S3 have to be closed. All these configurations have in common that the converter voltage is zero (0 state).

FIG. 4 shows the situation when all four switches S1, S2, S3 and S4 are in the off-state. In this situation the direction of the DC input voltage is inverted and it is possible for the source of the DC input voltage 401, 408 to gain electrical energy back. An advantage according to this circuit is the possibility to save electrical energy by regaining electrical energy from the supplied circuit.

FIG. 4 shows the input voltage DC in 401 and 408 which is supplied to the DC-AC converter 402 and 403. The DC-AC converter 402 and 403 comprises the four switches S1, S2, S3 and S4 together with the free-wheeling diodes D1, D2, D3 and D4. The switched voltage of the DC-AC converter 402, 403 is supplied to the resonant circuit with the inductance 404 and the resonant capacitor 405 and a transformer 407, which is represented by a parasitic capacitor CP. The output voltage of the transformer 407 is supplied to the rectifier 406. In this situation of FIG. 4 all four switches S1, S2, S3 and S4 are in an off-state. The electric energy which is stored in the resonant circuit 404, the resonant capacitor 405 and the transformer 407 can lead to a current 409. The current 409 supplies the source of the DC input voltage 401 and 408 which leads to a recovery of electrical energy. Voltage and current are in anti-phase relation (− state).

FIG. 5 depicts a control device 504, which comprises a controller 503, a predictor 501 and a decision block 502. The control device 504 calculates the control strategy for controlling the switches S1, S2, S3 and S4 of the DC-AC converter. The predictor 501 predicts the change of the output voltage of the rectifier for the next control cycle given by the zero crossing of the resonant current Ires 209, 309, 409 for all possible switch configurations plus-level, zero-level and minus-level. The controller 503 calculates a required change of the output voltage for the next step. The decision block 502 decides which control mode will be used, by choosing the control mode where the resulting change of the output voltage is closest to the required value. The decision block 502 calculates the control value 507 by processing the output voltages of the predictor 515, 514 and 513 and by processing the output voltage of the controller 516. The input voltages of the controller 503 are a reference voltage 508 as well as an output voltage 505. The output voltage 505 is the output voltage of the rectifier cascade, which is supplied by the secondary side of the transformer. The controller 503 could be for example realized as a PI-controller. The resonant circuit Ires is the current 209, 309, 409 of the FIGS. 2, 3 and 4. The predictor 501 processes the output voltages 515, 514, 513 with the help of the input voltages Uout 505 and UC 506. The voltage UC 506 is the voltage over the capacitor C 205, 305, 405, 104. In a FIG. 5 there are depicted four interfaces 509, 510, 511, and 512. At the interface 511 there is the situation when the output voltage 505, the capacitor voltage 506 as well as the reference voltage 508 arrives at the control device 504. The interface 511 represents a first information content. At the interface 512 there is the situation between the predictor 501, the controller 503 and the decision block 502 and represents a second information content. The second information content is not reduced with respect to the first information content. The input voltages of the decision block 502 will be processed and lead to an information content at the interface 509. The information content at the interface 509 is reduced with respect to the information content at interface 512 and the information content at interface 511. At the interface 510 the information content is also reduced with respect to the information content of the interface 512 and the information content of the interface 511. Therefore, it is senseful to arrange a bottleneck of information transfer at the interfaces 509 or 510. A bottleneck of information transfer is the data transfer between the rotary part of the gantry and the stationary part of the gantry. Therefore, according to the inventive concept of the present invention the data transmission bottleneck of a computer tomography gantry should be arranged at the interfaces 509 or 510, because the information content at these interfaces 509 and 510 is reduced with respect to the interfaces 511 and 512.

FIG. 5 shows a controller 504, comprises a conventional PI controller 503. The output of the PI controller 503 is a desired value for the next output voltage step Δu_{out,ref} 516. A predictor 501 estimates the resulting output voltage step for each of the three operation modes. This prediction can be made on the basis of an analytical dynamical model that has been derived. However, the model has not to be quite accurate. An approximation of the exact model is sufficient, as the resulting three-level controller is very robust to tolerances of the system parameter. Finally a decision block 502 chooses the operation mode that causes to the output voltage step, that is closest to the desired output voltage Δu_{out,ref} 516 calculated by the PI controller 503.

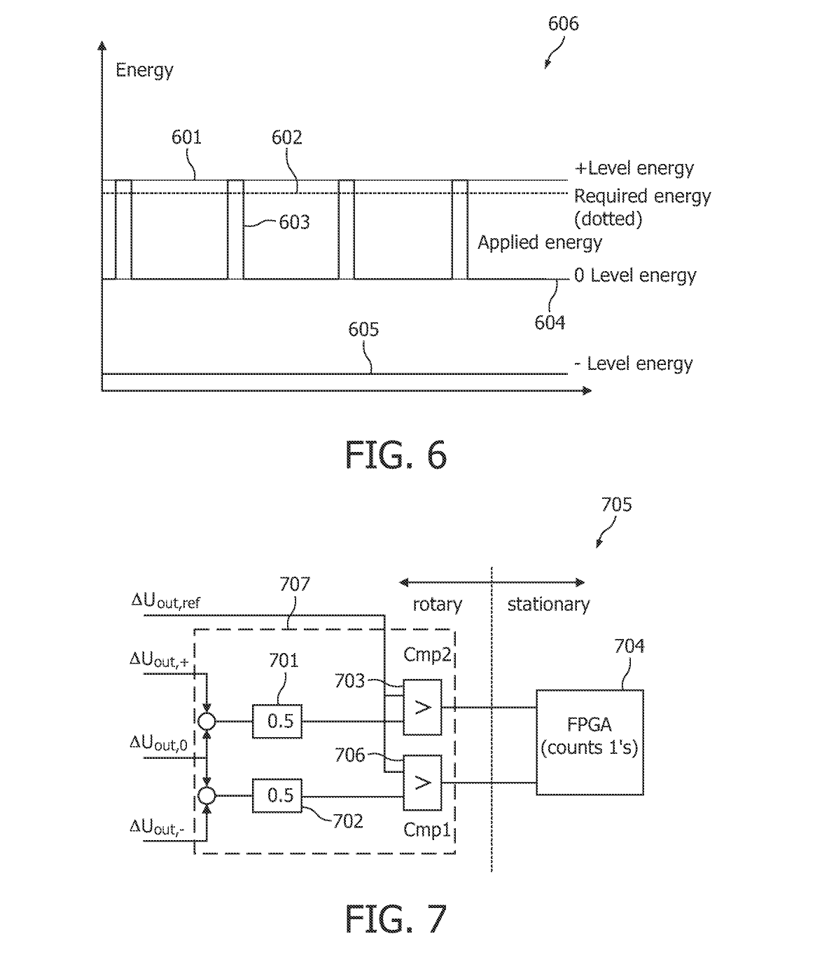

FIG. 6 shows the energy during the time. The dotted line 602 depicts the required energy which represents the output voltage of the rectifier 106. The line 603 depicts the output voltage of the DC-AC converter 102. The line 603 can have two levels. The first level 601 corresponds to the situation that energy is supplied to the circuit. In this case the line 603 is identical to the line 601. The line 601 represents the plus level energy. In case no power is supplied to the transformer the line 603 is identical to the line of the 0 level energy 604. In a situation as depicted in FIG. 4 a minus level energy 605 has to be regarded. As a result of the required energy with respect of the energy required by the transformer and the energy which is gained back because of the special arrangement of elements a required energy results, which is depicted with a dotted line 602.

The plus level energy 601 is a situation, which is realized by the situation of FIG. 2, the 0 level energy 604 is realized by a situation as depicted in FIG. 3, the minus level energy 605 is a situation which is realized by the situation as depicted in FIG. 4. In this FIG. 6 only the plus level energy 601 and the 0 level energy 604 is applied. Therefore, the line 603 changes between the line 601 and the line 604. In case a situation as depicted in FIG. 4 is achieved the line 603 would be identical for this time with the line 605. During the time duration of a situation as depicted in FIG. 4 the line of the applied energy 603 would be identical with the line of minus level energy 605.

FIG. 6 shows three energy levels 601, 604, 605, wherein the required energy for a specific operation point lies between the + and 0 level energy 601, 604. For a certain duration of time the +level energy 601 is activated and for another duration of time the 0 level energy is activated. Nevertheless the average energy in time is identical to the required energy. The result of applying the two different energy levels 601, 604 is a variation of the output voltage from the required voltage with a certain frequency and amplitude depending on the operation point. This phenomenon is called chattering.

Due to the described operation principle the chattering is in particular very sensitive to a delay in the measurement chain. Optimal is a delay in the range of 200 ns. Longer delays lead to considerably increased voltage variations (chattering). This problem can be solved by minimizing the amount of data that has to be transferred from the rotary part of the gantry to the stationary part of the gantry, in order to allow for a fast contactless data link.

FIG. 7 shows a block diagram of an embodiment of the invention. It is depicted an embodiment of the decision block 707. The input ΔUout,+, ΔUout,0, ΔUout,− are processed by an unit for calculating the medium value. These results will be compared by two comparators 703, 706 with the result of the closed loop ΔUout,ref the result thereof is transmitted to the logic unit. The logic unit could be a FPGA. After the processing of the decision block 707 the result of the processing is transmitted to the FPGA 704. Therefore, data is transmitted from the rotary part of the gantry to the stationary part of the gantry. The transmitted data content is reduced with respect to the data which comprises the input of the decision block 707. The input of the decision block 707 comprises ΔUout,ref, ΔUout,+, ΔUout,0 and ΔUout,−.

FIG. 7 depicts an embodiment of the controller according to FIG. 5. The decision block 707 is realized by two comparators 703, 706, which compare the medium value of Δu_{out,+} and Δu_{out,0} as well as the medium value of Δu_{out,0} and Δu_{out,-} with the output of the PI controller 503 Δu_{out,ref}. These comparators 703, 706 are placed on the rotary part of the gantry. The outputs (2 digital bits) of the comparators 703, 706 are then transferred to the stationary part of the gantry via a fast contactless data link. The FPGA 704 on the stationary part of the gantry simply counts the number of active comparators 703, 706. According to this embodiment the decision block 707 is arranged at the rotary part of the gantry. The FPGA 704 is arranged at the stationary part of the gantry. According to the invention it is necessary to arrange at least a part of the decision block 707 at the rotary part of the gantry.

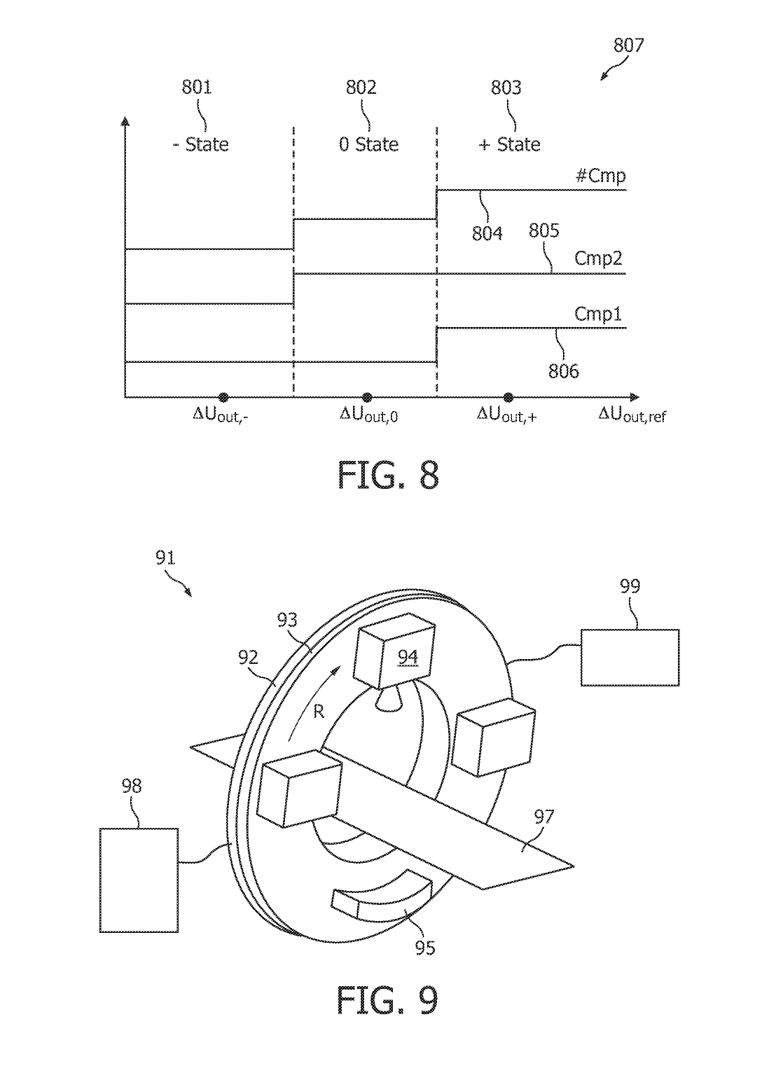

FIG. 8 shows the different output voltages of the comparators 703, 706 as a function of Δu_{out,ref}. The comparator 706 has a voltage characteristic 806. The comparator 703 has a voltage characteristic 805. The selection of the states depends on the comparator outputs 805, 806.

FIG. 8 depicts the logic of the FPGA 704. If both comparators 703, 706 are active a + state 803 results. If only one comparator 703, 706 is active a 0 state 802 results. If no comparator 703, 706 is active the − state 801 results.

The decision block 707 can also be generalized for a 5-level controller. In this case 4 comparators will be used and the data link has to transfer 4 bits (in contrast to the 2 digital bits in the embodiment shown in FIG. 7). However, these 4 bits can be reduced to three bits as there are only 5 different control states.

FIG. 9 shows an exemplary embodiment of a computer tomography gantry 91 arrangement. The gantry 91 comprises a stationary part 92 connected to a high frequency power source 98 and a rotary part 93 adapted to rotate relative to the stationary part 92. An X-ray source 94 and an X-ray detector 95 are attached to the rotary part 93 at opposing locations such as to be rotatable around a patient positioned on a table 97. The X-ray detector 95 and the X-ray source 94 are connected to a control and analysing unit 99 adapted to control the X-ray detector 95 and the X-ray source and to evaluate the detection results of the X-ray detector 95.

It should be noted that the term ‘comprising’ does not exclude other elements or steps and the ‘a’ or ‘an’ does not exclude a plurality. Also elements described in association with the different embodiments may be combined.

It should be noted that the reference signs in the claims shall not be construed as limiting the scope of the claims.

### LIST OF REFERENCE SIGNS

91 Computer tomography gantry,
92 Stationary part of the gantry,
93 Rotary part of the gantry,
94 X-ray source,
95 X-ray detector,
97 Table,
98 High frequency power source,
99 Control and analysing unit.
101 DC input voltage,
102 DC-AC converter,
103 Resonant inductance,
104 Resonant capacitor,
105 Transformer,
106 Rectifier,
107 High-voltage output,
108 Output voltage,
201 DC input voltage,
202 DC-AC converter,
203 DC-AC converter,
204 Resonant inductance,
205 Resonant capacitor,
206 Rectifier,
207 Parasitic capacitor,
208 DC input voltage,
209 Resonant current,
301 DC input voltage,
302 DC-AC converter,
303 DC-AC converter,
304 Resonant inductance,
305 Resonant capacitor,
306 Rectifier,
307 Parasitic capacitor,
308 DC input voltage,
309 Resonant current
401 DC input voltage,
402 DC-AC converter,
403 DC-AC converter,
404 Resonant inductance,
405 Resonant capacitor,
406 Rectifier,
407 Parasitic capacitor,
408 DC input voltage,
409 Resonant current,
501 Predictor,
502 Decision block,
503 Controller,
504 Control device,
505 Uout,
506 UC,
507 Control value,
508 Uref,
509 Information interface,
510 Information interface,
511 Information interface,
512 Information interface,
513 ΔUout,−
514 ΔUout,0
515 ΔUout,+
516 ΔUout,ref
601 Plus level energy,
602 Required energy,
603 Applied energy,
604 0 level energy,
605 Minus level energy,
701 Unit for calculating medium value,
702 Unit for calculating medium value,
703 Comparator,
704 Logic unit,
705 Embodiment of a decision block,
706 Comparator,
707 Decision block,
801 Minus state,
802 0 state,
803 Plus state,
804 Voltage characteristic of a comparator,
805 Voltage characteristic of the comparator 2,
806 Voltage characteristic of the comparator 1,
807 Mode of operation of a decision block.

1. An X-ray device for controlling a DC-AC converter, wherein the DC-AC converter is adapted for supplying a resonant circuit and a transformer (105) of a computer tomography gantry (91) with electrical energy, wherein
the gantry (91) comprises
a rotary part (93) and
a stationary part (92),
wherein the transformer (105) is adapted for providing a current, feeding a high voltage rectifier circuit (106), providing an output voltage (107),
the X-ray device comprises
a detector for detecting the output voltage,
a predictor (501) for calculating a first output with the use of processing the output voltage (107), wherein the first output represents the change of the output voltage (107) for the possible states of the DC-AC converter (102),
a control loop (503) for calculating the required change of the output voltage (107) with the use of processing the output voltage (107) and the target specification,
a decision block (502) for calculating a control value with the use of processing the first output and the required change of the output voltage (107),
wherein the detector, the predictor (501), the control loop (503) and at least a part of the decision block (502) are adapted to be mounted on the rotary part of the gantry, such as the information content to be transmitted from the rotary part of the gantry to the stationary part of the gantry is less than the information content of the output voltage.

2. The X-ray device according to claim 1, wherein the control loop (503) is a PI-control loop.

3. The X-ray device according to claim 1, wherein the decision block (502) is adapted to calculate the control value to control the DC-AC converter (102).

4. The X-ray device according to claim 1, further comprising
a logic unit (704) for controlling the DC-AC converter (102), wherein the decision block (502) is adapted to control the logic unit (704) by the control value.

5. The X-ray device according to claim 4, wherein the logic unit (704) is programmable.

6. The X-ray device according to claim 5, wherein the logic unit (704) is a FPGA or a CPLD.

7. The X-ray device according to claim 4, wherein the logic unit (704) is adapted to be mounted on the stationary part of the gantry (92).

8. The X-ray device according to claim 1, wherein the predictor (501) is adapted for generating three predictions of the required change of the output voltage (107).

9. The X-ray device according to claim 1, wherein the predictor (501) is adapted for generating five predictions of the required change of the output voltage (107).

10. The X-ray device according to claim 1, wherein the resonant circuit comprises a resonance capacitor (104), wherein
the X-ray device comprises
a second detector for detecting the capacitor voltage over the resonance capacitor (104), wherein the predictor (501) is adapted for calculating a second output with the use of processing the capacitor voltage, which represents the change of the output voltage (107) for the possible states of the DC-AC converter (102).

11. A computer tomography gantry (91) comprising a X-ray device according to claim 1.
